(19) United States
(12) Patent Application Publication
(10) Pub. No.: US 2008/0260770 A1
(43) Pub. Date: Oct. 23, 2008

(54) Use of Tellurium Compounds as Adjuvants

(75) Inventors: Benjamin Sredni, Kfar-Saba (IL); Michael Albeck, Ramat-Gan (IL)

Correspondence Address: MARTIN D. MOYNIHAN d/b/a PRTSI, INC., P.O. BOX 16446, ARLINGTON, VA, 22215, US

(73) Assignee: BIOMAS LTD., Tel-Aviv (IL)

(21) Appl. No.: 11/662,954

(22) PCT Filed: Sep. 15, 2005
(86) PCT No.: PCT/IL05/00992

§ 371 Date: Mar. 15, 2007

Related U.S. Application Data
(60) Provisional application No. 60/610,660, filed on Sep. 17, 2004.
(60) Provisional application No. 60/686,966, filed on Jun. 3, 2005.

(51) Int. Cl. A61K 39/00; A61K 47/04; A61P 37/04; A61P 35/00; A61K 47/08

(52) U.S. Cl. 424/193.1; 424/278.1; 424/277.1

(57) ABSTRACT

Methods for enhancing the immune response of a subject to an immunoeffector, and methods of enhancing interleukin-12 production, which are effected by administering an amount of the immunoeffector and an effective adjuvanting amount of a tellurium-containing compound are provided. The enhanced immune response may be a cell-mediated or a humoral immune response. A pharmaceutical composition, which comprises the tellurium-containing compound, the immunoeffector and a pharmaceutically acceptable carrier is also provided. Use of a tellurium-containing compound as an adjuvant for immunization is also provided.

### FIELD AND BACKGROUND OF THE INVENTION

The present invention relates to tellurium-containing compounds and their use as adjuvants.

The immune system of higher organisms is comprised of an adaptive and an innate component. The innate immune system includes phagocytic cells, which includes macrophages and polymorphonuclear leukocytes that can engulf (phagocytose) foreign substances. The adaptive immune system is based on leukocytes, and is divided into two major sections: the humoral immune system, which acts mainly via immunoglobulins produced by B cells, and the cell-mediated immune system, which functions mainly via T cells.

Protective immunity induced by vaccination is dependent on the capacity of the vaccine to elicit the appropriate immune response to either resist, control, or eliminate the pathogen. Depending on the pathogen, this may require a cell-mediated or humoral immune response, which, in turn, is determined by the nature of the T cells that develop after immunization. These are comprised of two major subsets: T_{H1} that produce interleukin-2 (IL-2) and interferon-γ (IFN-γ), and are involved in cell-mediated responses; and T_{H2} that produce IL-4, IL-5, and IL-10 and augment humoral immune responses.

Non-protein antigens such as polysaccharides and lipids induce antibody responses without the need for T cells and are therefore referred to as T-independent (TI) antigens. However, because of the lack of involvement of T cell help, most TI antigens are relatively poor immunogens. In general, responses to TI antigens consist of IgM antibodies of low affinity, and do not show significant heavy chain class switching, affinity maturation, or memory. The practical significance of TI antigens is that many bacterial capsular and cell wall polysaccharides belong to this category and are therefore relatively poor at eliciting humoral immunity

Chemotherapeutic agents generally work by impairing mitosis, effectively targeting fast-dividing cells. The majority of chemotherapeutic drugs can be divided into alkylating agents, anti-metabolites, plant alkaloids, topoisomerase inhibitors and antitumor agents, all of which affect cell division or DNA synthesis.

An adjuvant is a substance which enhances the immune-stimulating properties of an immunoeffector, such as an antigen, by co-stimulating the immune system when a vaccine or a chemotherapeutic agent is given. Adjuvants have the capability of influencing antibody titer, response duration, isotype, avidity and some properties of cell-mediated immunity. The use of adjuvants is required for many antigens which by themselves are weakly immunogenic. Their mode of action is either non-specific, resulting in increased immune responsiveness to a wide variety of antigens, or antigen-specific, i.e., affecting a restricted type of immune response to a narrow group of antigens. The therapeutic efficacy of many biological response modifiers is related to their antigen-specific immuno-adjuvanticity.

Adjuvants may act through a number of different mechanisms. One mechanism involves enhancing long term release of the antigen by functioning as a depot. Long term exposure to the antigen increases the length of time the immune system is presented with the antigen for processing as well as the duration of the antibody response. Another mechanism is by interaction with immune cells. Adjuvants may act as non-specific mediators of immune cell function by stimulating or modulating immune cells. In brief, it is believed that adjuvants bind to specific receptors on the surfaces of macrophages, resulting in stimulation of the maturation of the macrophages by inducing these macrophages to make and release type-1 interferons. These type-1 interferons interact with receptors on the surface of other macrophages, activating them, or the type-1 interferons interact with the same macrophage that produced them, and activate it. In either case, the activated macrophages then begin to express essential “costimulatory” molecules like CD80, CD86, and CD40 that finally activate T-cells of the adaptive immune system, and the active T cells produce a highly specific immune response against the immunoeffector. Adjuvants may also enhance macrophage phagocytosis after binding the antigen as a particulate (a carrier/vehicle function).

The choice of adjuvant is exceedingly important from both the aspect of the end result (high antibody response) as well as the immunized subject's welfare. Many potential adjuvants have the capacity to cause inflammation, tissue necrosis and pain in the recipient. Selection of an adjuvant is based upon antigen characteristics (size, net charge and the presence or absence of polar groups).

Currently available adjuvants include:
Complete Freund's Adjuvant (CFA): A mineral oil adjuvant; which uses a water-in-oil emulsion which is primarily oil. This adjuvant, while potent immunogenically, has been found to frequent produce abscesses, granulomas and tissue sloughs. It contains paraffin oil, killed mycobacteria and mannide monoosleate. The paraffin oil is not metabolized; it is either expressed through the skin (via a granuloma or abscess) or phagocytized by macrophages. Multiple exposures to CFA will cause severe hypersensitivity reactions. Accidental exposure of personnel to CFA may result in sensitization to tuberculin.
Incomplete Freund's Adjuvant (IFA): Also a mineral oil adjuvant, having a composition similar to that of CFA but does not contain the killed mycobacteria so does not produce as severe reactions. IFA is used for the booster immunizations following the initial injection with antigen-CFA. May be used for initial injection if the antigen is strongly immunogenic.
Montanide ISA (incomplete seppic adjuvant): A mineral oil adjuvant, which uses mannide oleate as the major surfactant component. The antibody response is generally similar to that with IFA. May have a lessened inflammatory response.
Ribi Adjuvant System (RAS): An oil-in-water emulsion that contains detoxified endotoxin and mycobacterial cell wall components in squalene. RAS has lower viscosity than CFA, and produces titers which are often comparable to those with CFA. The squalene oil is metabolizable. Lower incidence of toxic reactions.
TiterMax: Another water-in-oil emulsion, which combines a synthetic adjuvant and microparticulate silica with squalene. The copolymer is the immunomodulator component. Antigen is bound to the copolymer and presented to the immune cells in a highly concentrated form. Less toxicity than CFA. Usually produces the same results as CFA.
Syntex Adjuvant Formulation (SAF): A preformed oil-in-water emulsion. Uses a block copolymer for a surfactant. A muramyl dipeptide derivative is the immunostimulatory component. All in squalene, a metabolizable oil. May bias the humoral response to IgG2a in the mouse. Less toxic than CFA.
Aluminum Salt Adjuvants: These are the only adjuvant approved for use in the United States for human vaccines. Generally weaker adjuvants than emulsion adjuvants. Best used with strongly immunogenic antigens. Generally mild inflammatory reactions.
Nitrocellulose-adsorbed antigen: The nitrocellulose is basically inert, leading to almost no inflammatory response. Slow degradation of nitrocellulose paper allows prolonged release of antigen. Does not produce as dramatic an antibody response as CFA.
Encapsulated or entrapped antigens: Permits prolonged release of antigen over time; may also have immunostimulators in preparation for prolonged release. Preparation is complex.
Immune-stimulating complexes (ISCOMs): Antigen modified saponin/cholesterol micelles. Stable structures are formed which rapidly migrate to draining lymph nodes. Both cell-mediated and humoral immune responses are achieved. Low toxicity; may elicit significant antibody response.
Gerbu® adjuvant: An aqueous phase adjuvant. Uses immunostimulators in combination with zinc proline. Does not have a depot effect. Minimal inflammatory effect. Requires frequent boosting to maintain high titers.

Many of the most effective adjuvants include bacteria or their products. It has been shown that bacterial adjuvants function by production of Interleukin-12 (IL-12), which results in enhanced development of T helper cells (Science, 260: 547, 1993). However, despite their immunostimulating properties, many bacterial adjuvants have toxic or other negative effects.

IL-12 is a heterodimeric cytokine which is naturally produced by macrophages and B lymphocytes in response to antigenic stimulation. It has been found to stimulate the production of IFN-γ from T and natural killer (NK) cells (Science 260:496, 1993; (Kobayashi et al., J. Exp. Med, 170:827, 1989), to promote NK activity, and enhance CTL maturation (Germann, et al., Eur. J. Immmunol. 23:1762, 1993). IL-12 induces Th1-type immune responses by activating maturation of type 1 Th cells from an uncommitted T cell pool.

In cancer patients, IL-12 has an antitumor effect based on several mechanisms: the activation of innate and antigen-specific adaptive immunity against tumor cells and the ability to inhibit tumor angiogenesis through INF-γ (Trinchieri, G., Interleukin 12. In: Theze J (ed.) The cytokine network and immune functions. Oxford university press, Oxford, p 97-103). IL-12 is also known as an endogenous inhibitor of angiogenesis (Toi et al. 1999). Moreover, intratumor injection of an adenoviral vector with IL-12 has been suggested to have anti-angiogenic effects enhancing the local and anti-tumor effects of irradiation (Seetharam et al., Int. J. Oncol. 15: 769, 1999).

Protective adjuvant effects of IL-12 protein co-administration have been observed in mouse bacterial infection models (Miller, et al., Ann. NY Acad. Sci. 797:207, 1996). When used as a molecular adjuvant, IL-12 cDNA induces Ag-specific CTL responses with inhibitory effects on humoral responses in HIV DNA vaccine studies (Kim, et al., J. Immunol 158:816, 1997). Iwasaki et al. (J. Immunol. 158:4591, 1997) similarly reported that IL-12 cDNA co-delivered with DNA encoding for influenza NP resulted in enhanced cellular immune responses.

U.S. Pat. Nos. 5,723,127; 5,976,539; 6,071,893; 6,168,923; and 6,303,114, as well as Science, 263:235, 1994, which are all incorporated by reference as if fully set forth herein, disclose use of recombinant IL-12 as an adjuvant in various immunization applications.

Use of recombinant IL-12 involves a complex preparation process, which requires expression and isolation of IL-12 protein in recombinant cell hosts. The prior art, however, does not teach the use of an adjuvant which stimulates natural production of IL-12 by the immune system.

There is thus a widely recognized need for and it would be highly advantageous to have novel adjuvants, devoid of the above limitations.

Various tellurium compounds have been described in the art as having immunomodulating properties. A particularly effective family of tellurium-containing compounds is taught, for example, in U.S. Pat. Nos. 4,752,614; 4,761,490; 4,764,461 and 4,929,739, whereby another effective family is taught, for example, in a recently filed U.S. Provisional Patent Application No. 60/610,660, which are all incorporated by reference as if fully set forth herein. The immunomodulating properties of this family of tellurium-containing compounds is described, for example, in U.S. Pat. Nos. 4,962,207, 5,093,135, 5,102,908 and 5,213,899, which are all incorporated by reference as if fully set forth herein.

One of the most promising compounds described in these patents is ammonium trichloro(dioxyethylene-O,O′)tellurate, which is also referred to herein and in the art as AS101. AS101, as a representative example of the family of tellurium-containing compound discussed hereinabove, exhibits antiviral (Nat. Immun. Cell Growth Regul. 7(3):163-8, 1988; AIDS Res Hum Retroviruses. 8(5):613-23, 1992), and tumoricidal activity (Nature 330(6144):173-6, 1987; J. Clin. Oncol. 13(9):2342-53, 1995; J Immunol 161(7):3536-42, 1998.

It has been suggested that AS101, as well as other tellurium-containing immunomodulators, stimulate the innate and acquired arm of the immune response. For example, it has been shown that AS101 is a potent activator of interferon (IFN) (IFN) in mice (J. Natl. Cancer Inst. 88(18):1276-84, 1996) and humans (Nat. Immun. Cell Growth Regul. 9(3):182-90, 1990; Immunology 70(4):473-7, 1990; J. Natl. Cancer Inst. 88(18):1276-84, 1996.)

It has also been demonstrated that AS101, as well as other tellurium-containing immunomodulators, induce the secretion of a spectrum of cytokines, such as IL-1, IL-6 and TNF-α, and that macrophages are one main target for AS101 (Exp. Hematol. 23(13):1358-66, 1995) and it was found to inhibit IL-10 at the m-RNA level, and this inhibition may cause an increase in IL-112 (Cell Immunol. 176(2):180-5, 1997); J. Natl. Cancer Inst. 88(18):1276-84, 1996).

Other publications describing the immunomodulation properties of AS101 include, for example, “The immunomodulator AS101 restores T(H1) type of response suppressed by Babesia rodhaini in BALB/c mice”. Cell Immunol 1998 February; “Predominance of TH1 response in tumor-bearing mice and cancer patients treated with AS101”. J Natl Cancer Inst 1996 September; “AS-01: a modulator of in vitro T-cell proliferation”. Anticancer Drugs 1993 June; “The immunomodulator AS101 administered orally as a chemoprotective and radioprotective agent”. Int J Immunopharmacol 1992 May; “Inhibition of the reverse transcriptase activity and replication of human immunodeficiency virus type 1 by AS101 in vitro”. AIDS Res Hum Retroviruses 1992 May; “Immunomodulatory effects of AS101 on interleukin-2 production and T-lymphocyte function of lymphocytes treated with psoralens and ultraviolet A”. Photodermatol Photoimmunol Photomed 1992 February; “Use and mechanism of action of AS101 in protecting bone marrow colony forming units-granulocyte-macrophage following purging with ASTA-Z 7557”. Cancer Res 1991 Oct. 15; “The effect of the immunomodulator agent AS101 on interleukin-2 production in systemic lupus erythematosus (SLE) induced in mice by a pathogenic anti-DNA antibody”. Clin Exp Immunol 1990 March; “Toxicity study in rats of a tellurium based immunomodulating drug, AS-101: a potential drug for AIDS and cancer patients”. Arch Toxicol 1989; “The biological activity and immunotherapeutic properties of AS-101, a synthetic organotellurium compound”. Nat Immun Cell Growth Regul 1988; and “A new immunomodulating compound (AS-101) with potential therapeutic application”. Nature 1987 November.

In addition to its immunomodulatory effect, AS101 is also characterized by low toxicity. Toxicity tests have shown that LD50 values in rats following intravenous and intramuscular administration of AS101 are 500-1000 folds higher than the immunologically effective dose.

While the immunomodulating effect of tellurium-containing compounds was studied with respect to various aspects thereof, the use of tellurium compounds as adjuvants has never been suggested nor practiced hitherto.

### SUMMARY OF THE INVENTION

According to one aspect of the present invention there is provided a method for enhancing the immune response of a subject to an immunoeffector, the method comprising administering to the subject an amount of the immunoeffector and an effective adjuvanting amount of at least one tellurium-containing compound.

The immune response enhanced by the method of the present invention may be a cell-mediated response, or a humoral immune response, such as that resulting in an enhanced IgG2a antibody response.

According to another aspect of the present invention, there is provided a use of a tellurium-containing compound as an adjuvant for immunization, namely, for enhancing the immune response of a subject to an immunoeffector.

According to further features in preferred embodiments of the invention described below, the tellurium-containing compound comprises at least one tellurium dioxide moiety and, optionally and preferably, is at least one of tellurium dioxide (TeO₂), a complex of TeO₂, a compound having general Formula I:

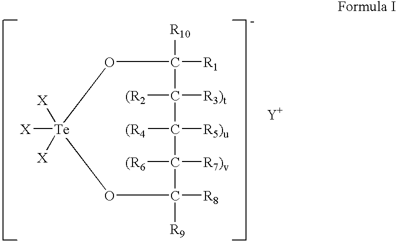

a compound having general Formula II:

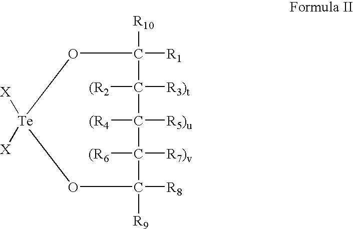

a compound having general Formula III:

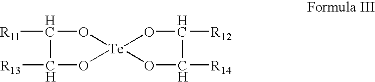

and
a compound having general Formula IV:

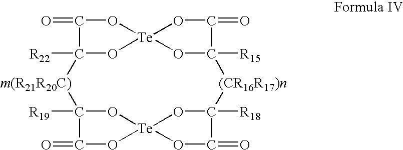

wherein:

each of t, u and v is independently 0 or 1;

each of m and n is independently an integer from 0 to 3;

Y is selected from the group consisting of ammonium, phosphonium, potassium, sodium and lithium;

X is a halogen atom; and

each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido.

Preferably, the tellurium-containing compound has general Formula I or general Formula IV.

According to an embodiment in which the tellurium-containing compound has general Formula I, preferably t, u and v are each 0. More preferably, each of R₁, R₈, R₉ and R₁₀ is hydrogen; more preferably X is a halogen atom, most preferably the halogen atom is chloro. More preferably, Y is ammonium. The preferred compound according to this embodiment is referred to hereinafter as AS101.

According to an alternative embodiment of this feature of the present invention, the tellurium-containing compound has the general Formula IV. Preferably, according to this embodiment, n and m are each 0. More preferably, each of R₁₅, R₁₈, R₁₉ and R₂₂ is hydrogen. The preferred compound according to this embodiment is referred to hereinafter as SAS.

Preferably, the immunoeffector of the present invention is an antigen, which may be either a T-cell dependent or a T-cell independent antigen. More preferably, the antigen is derived from a pathogenic microorganism, including, but not limited to, an intracellular parasite; a virus, such as HIV, Hepatitis A, Hepatitis B, Hepatitis C, rabies virus, Herpes viruses, Cytomegalovirus, poliovirus, influenza virus, meningitis virus, measles virus, mumps virus, rubella, varicella, pertussis, encephalitis virus, papilloma virus, yellow fever virus, Epstein-Barr virus, respiratory syncytial virus, parvovirus, chikungunya virus, haemorrhagic fever viruses, and Klebsiella; a virus of the paramyxoviridae family, including human paramyxoviridae viruses such as paramyxoviruses (e.g. parainfluenza virus 1, parainfluenza virus 2, parainfluenza virus 3 and parainfluenza virus 4), morbilliviruses (e.g. measles virus) and pneumoviruses (e.g., respiratory syncytial virus); a non-human paramyxoviridae virus, such as canine distemper virus, bovine respiratory syncytial virus, Newcastle disease virus and rhinderpest virus; or an extracellular parasite, such as a bacterium, a protozoan (such as babesia), and a helminth, including extracellular parasites which cause leprosy, tuberculosis, leishmania, malaria, or schistosomiasis.

According to an embodiment of the present invention in which the immunoeffector is a T-cell independent antigen, enhancement of the immune response comprises enhancing a T-cell independent immune response to the T-cell independent antigen.

Representative examples of T-cell independent antigens that can be used as immunoeffectors according to this embodiment of the present invention include, without limitation, carbohydrates (e.g. polysaccharides, lipopolysaccharides), lipids (e.g. liposomes), glycolipids, phosopholipids (e.g. phosphorylcholine), carrier conjugates (e.g. H. influenza conjugate vaccine, polysaccharide conjugate, lipid conjugate, phage conjugate), viruses (e.g., phages, such as T4 phage), parasites, fungi and yeast, and TI antigens recognized by immature T cells (e.g., CD1 molecules), such as lipoarabinomannan., which when administered to a subject activate the immune response without interacting with the T-lymphocytes.

Polysaccharides which may be used as immunoeffectors according to this embodiment of the present invention include, without limitation, bacterial polysaccharides, such as bacterial capsular polysaccharides (e.g., Streptococcus pneumoniae capsular polysaccharide, such as the PNU-Immune 23 vaccine, Neisseria meningiditis A, C, Y and W-135 serogroups, Haemophilus influenzae, Brucella abortis), and bacterial gram-negative cell wall polysaccharides.

According to an embodiment of the present invention in which the antigen is a T-cell dependent antigen, enhancement of the immune response comprises enhancing a type 1/Th1 T cell immune response. Preferably, the antigen is a lipopeptide. The lipopeptide may be, for example, a lipoprotein of Mycobacterium tuberculosis

According to a further preferred embodiment of the present invention, the immunoeffector is a chemotherapeutic agent.

According to an alternative embodiment of the present invention, the immunoeffector is an antigen derived from a cancer cell or a cancer cell transfected with a selected antigen. In accordance with this embodiment, enhancement of the immune response comprises eliciting a cell mediated immune response in the subject against the cancer cell or the cancer cell transfected with the selected antigen.

According to still another aspect of the present invention there is provided a method of enhancing interleukin-12 production in a subject having a condition in which enhanced immune response induced by an immunoeffector is beneficial, the method comprising administering to the subject an effective adjuvanting amount of at least one tellurium-containing compound. Such conditions include, for example, cancer, including leukemia and solid tumors, such as adrenal tumors, bone tumors, gastrointestinal tumors, brain tumors, breast tumors, skin tumors, lung tumors, ovarian tumors, and genitourinary tumors; an immune deficiency, such as HIV positive or Acquired Immunodeficiency Syndrome (AIDS); an autoimmune disease; a viral disease; and an infectious disease.

The compounds of the present invention may be administered by any suitable route, such as the oral, rectal, transmucosal, intestinal, parenteral, intrathecal, direct intraventricular, intravenous, inrtaperitoneal, intranasal, and intraocular routes.

In any of the methods described herein, the tellurium-containing compound preferably forms a part of a pharmaceutical composition, as described hereinbelow.

Thus, according to a further aspect of the present invention there is provided a pharmaceutical composition, which comprises the tellurium-containing compound, the immunoeffector and a pharmaceutically acceptable carrier.

In embodiments wherein the tellurium-containing compound has general Formula I or general Formula IV, the concentration of the tellurium-containing compound preferably ranges from about 0.1 to about 20 μg per 1 ml of the carrier, more preferably from about 0.2 to about 10 μg per 1 ml of the carrier. Alternatively, the concentration of the tellurium-containing compound preferably ranges from about 0.01 weight percent to about 50 weight percents of the total weight of the composition.

In embodiments wherein the tellurium-containing compound has general Formula I, and wherein t, u and v are each 0, each of R₁, R₈, R₉ and R₁₀ is hydrogen, X is chloro, and Y is ammonium, a concentration of the tellurium-containing compound preferably ranges from about 0.1 μg to about 10 μg per 1 ml carrier, more preferably from about 0.2 μg to about 5 μg per 1 ml carrier and more preferably from about 0.5 μg to about 2 μg per 1 ml carrier.

In embodiments wherein the tellurium-containing compound has general Formula IV, wherein n and m are each 0, and wherein each of R₁₅, R₁₈, R₁₉ and R₂₂ is hydrogen, the concentration of the tellurium-containing compound preferably ranges from about 0.1 μg to about 20 μg per 1 ml of the carrier, more preferably from about 0.5 μg to about 10 μg per 1 ml of carrier and more preferably from about 0.8 μg per 1 ml of carrier to about 4 μg per 1 ml of carrier.

The present invention successfully addresses the shortcomings of the presently known adjuvants by providing novel adjuvants, which are highly efficient, and induce minimal or no adverse side effects.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “therapeutically effective amount” or “pharmaceutically effective amount” denotes that dose of an active ingredient or a composition comprising the active ingredient that will provide the therapeutic effect for which the active ingredient is indicated.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph demonstrating the effect of AS101 on IL-12 production by human monocytes;

FIG. 2 is a bar graph demonstrating the effect of AS101 on IL-12p40 production by murine bone marrow-derived dendritic cells; and

FIGS. 3a-b are bar graphs demonstrating serum antibody responses to depyrogenated keyhole limpet hemocyanin (KLH), as determined by titers of KLH-specific IgG1 (FIG. 3a) and IgG2a (FIG. 3b).

### DESCRIPTION OF THE PREFERRED EMBODIMENTS

The present invention is of novel adjuvants that comprise tellurium-containing compounds, which can be efficiently used for enhancing the immune-stimulating properties of an immunoeffector.

The principles and operation of the compositions and methods according to the present invention may be better understood with reference to the accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Presently known and/or utilized adjuvants are limited by toxic and allergenic effects, or are extremely expensive to produce.

As is described hereinabove, prior art studies have shown a relationship between IL-12 and enhancement of the immunogenic effect of a vaccine or a chemotherapeutic agent. These studies involved the use of recombinant IL-12, which is expensive and complicated to produce. Prior art studies have also conclusively shown that tellurium-containing compounds function as immunomodulators, and thus can be used in treatment of cancer, immune deficiencies, autoimmune diseases and infectious diseases.

While conceiving the present invention, the present inventors have postulated that the immunomodulatory effect of the tellurium containing compound AS101 can be harnessed to enhance the immune response to an administered antigen or other immunoeffector.

As is demonstrated in the Examples section that follows, experiments conducted by the present inventors while reducing the present invention to practice conclusively show that tellurium-containing compounds such as AS101 are capable of inducing IL-12 production in immune cells such as monocytes and bone marrow-derived dendritic cells. These experiments further demonstrated that AS101 increases serum IgG2a antibody production.

These results therefore demonstrate that AS101 has an immune stimulatory effect and indicate that using tellurium-containing compounds such as AS101 and related compounds would beneficially result in increased immune and chemotherapeutic responsiveness to processes mediated by IL-12, with no or minimized adverse side effects.

Thus, according to one aspect of the present invention there is provided a method of enhancing the immune response of a subject to an immunoeffector. The method, according to this aspect of the present invention, is effected by administering to the subject an amount of the immunoeffector and an effective adjuvanting amount of at least one tellurium-containing compound, as is detailed hereinunder.

As used herein, the term “immunoeffector” refers to a molecule, composition or cell which can potentially elicit an immune response in a subject administered thereto. It should be noted that since some antigens do not elicit an immune response when administered in the absence of an adjuvant or carrier, the term immunoeffector also encompasses molecules, compositions or cells which only elicit an immune response when co-administered with a carrier molecule or an adjuvant.

The immune response enhanced by the method according to this aspect of the present invention may be a cell-mediated response, or a humoral immune response which can result in an enhanced IgG2a antibody response.

As used herein, the phrase “enhancing” or “enhanced” regarding the immune response to an immunoeffector describes increasing, strengthening or inducing an immune response to the immunoeffector.

As used herein, the phrase “effective adjuvanting amount” refers to that amount of a compound which, when administered simultaneously or sequentially with an inimunoeffector, produces enhancement of the effect obtained with the immunoeffector alone or alternatively induces an immune response to the immunoeffector.

One of skill in the art is expected to be able to readily determine suitable amounts of the tellurium-containing compound to adjuvant certain immunoeffectors. Such amounts will typically depend upon the nature of the immunoeffector, the dosage amounts of the immunoeffector as well as the species and physical and medical conditions (e.g., general health, weight, etc.) of the subject. The amount of the immunoeffector can be similarly determined.

While depending upon the nature of the immunoeffector and the physical and medical condition of the subject, presently known immunoeffectors used for stimulating an immune response are typically administered in an amount that ranges from about 0.01 μg to about 50 μg per 1 ml carrier.

An adjuvanting effective amount of the tellurium-containing compounds described herein can range, for example, from about 0.01 mg/m² to about 20 mg/m² or from about 0.1 mg/m² to about 10 mg/m².

The tellurium-containing compound may be administered simultaneously or sequentially with the immunoeffector. When the tellurium-containing compound is administered simultaneously with the immunoeffector, both the immunoeffector and the tellurium-containing compound can form a part of the same composition. Such compositions are described in detail hereinunder.

Alternatively, the adjuvanting effect of the tellurium-containing compound may be employed by administering the tellurium-containing compound separately from the immunoeffector composition. When administered separately, the tellurium-containing compound is preferably provided in a suitable carrier, such as saline or PBS, and optionally conventional pharmaceutical agents. The tellurium-containing compound may be administered contemporaneously with the immunoeffector composition, or, alternatively, before or after the immunoeffector administration. The time interval between the administration of the immunoeffector and the tellurium-containing compound, which administered separately, typically depends on the immunoeffector employed and may range from one minute to a few hours.

Administration of the immunoeffector is effected according to the immunization schedule approved for the immunoeffector, such as that recommended by the US Department of Health and Social Services. When multiple administrations of the immunoeffector are desired, the tellurium-containing compound can be administered with the immunoeffector either only within the first administration or in all of the scheduled administrations.

Any route of administration may be employed for the administration of the immunoeffector, the tellurium-containing compound or a composition containing both, including oral, rectal, transmucosal, intestinal, parenteral, intrathecal, direct intraventricular, intramuscular, intravenous, intraperitoneal, intranasal, and intraocular administration. The immunoeffector and the tellurium-containing compound, when administered separately, can be administered either by the same route of administration or by different routes of administration.

The adjuvanting effect of the tellurium-containing compounds described herein may be utilized in this and other aspects of the present invention for enhancing the immune response to any of the presently known immunoeffectors, including those which enhance cell-mediated immune responses and those which enhance humoral immune responses.

The immunoeffector employed in this and any other aspect of the present invention can be obtained from a commercial source, or otherwise prepared using technologies well known in the art.

The immunoeffector can be, for example, an antigen or an antigen-presenting cell or composition. The antigen may be a T-cell dependent or T-cell independent antigen.

The antigen may be a whole cell, a protein, a protein subunit or fragment or a carbohydrate. Alternatively, DNA sequences encoding the antigen from a pathogenic microorganism, a subunit, or a fragment thereof, rather than the protein or peptide itself may be used. These DNA sequences, together with appropriate promoter sequences (as nucleic acid expression constructs), may be employed directly as the antigen. Any suitable promoter sequence can be used by such a nucleic acid construct, including promoters that are active in the specific cell population transformed. Examples of cell type-specific and/or tissue-specific promoters are described in Pinkert et al., (1987) Genes Dev. 1:268-277; Calame et al., (1988) Adv. Immunol. 43:235-275; Winoto et al., (1989) EMBO J. 8:729-733; Banerji et al. (1983) Cell 33729-740; Byrne et al. (1989) Proc. Natl. Acad. Sci. USA 86:5473-5477; Edlunch et al. (1985) Science 230:912-916; and in U.S. Pat. No. 4,873,316). Such nucleic acid constructs can further include an enhancer, which can be adjacent or distant to the promoter sequence and can function in up regulating the transcription therefrom, a cis acting regulatory element and/or an appropriate selectable marker and/or an origin of replication. The construct can be, for example, a plasmid, a bacmid, a phagemid, a cosmid, a phage, a virus or an artificial chromosome.

When a nucleic acid expression construct is utilized as the antigen, the tellurium-containing compound is preferably administered separately and after the expression of the protein from the construct.

Representative examples of antigens that can be used as immunoeffectors according to this embodiment of the present invention include, without limitation, antigens that are derived from pathogenic microorganisms.

The pathogenic microorganism can be, for example, an intracellular parasite, an extracellular parasite (such as a bacterium, a protozoa, and a helminth, for example those which cause leprosy, tuberculosis, leishmania, malaria, or schistosomiasis) or a virus.

Representative examples of viruses from which an antigen according to this embodiment of the present invention can be derived include, without limitation, HIV, Hepatitis A, Hepatitis B, Hepatitis C, rabies virus, Herpes viruses, Cytomegalovirus, poliovirus, influenza virus, meningitis virus, measles virus, mumps virus, rubella, varicella, pertussis, encephalitis virus, papilloma virus, yellow fever virus, Epstein-Barr virus, respiratory syncytial virus, parvovirus, chikungunya virus, haemorrhagic fever viruses, Klebsiella, a virus of the paramyxoviridae family, including human paramyxoviridae viruses such as paramyxoviruses (e.g. parainfluenza virus 1, parainfluenza virus 2, parainfluenza virus 3 and parainfluenza virus 4), morbilliviruses (e.g. measles virus) and pneumoviruses (e.g., respiratory syncytial virus); a non-human paramyxoviridae virus, such as canine distemper virus, bovine respiratory syncytial virus, Newcastle disease virus and rhinderpest virus.

Additional examples of pathogenic microorganisms from which an antigen according to this embodiment of the present invention can be derived include gram negative bacteria, including but not limited to, Escherichia coli, Enterobacter aerogenes, Kiebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Flavobacterium spp., Haemophilus influenzae, Pseudomonas aeruginosa, Campylobacter jejuni, Vibrio parahaemolyticus, Brucella spp., Neisseria meningitidis, Neisseria gonorrhoea, Bacteroides fragilis, and Fusobacterium spp.

Alternatively, pathogenic microorganisms from which an antigen according to this embodiment of the present invention can be derived include Gram-positive bacteria, including but not limited to, Strep. pyogenes (Group A), Strep. pneumoniae, Strep. GpB, Strep. viridans, Strep. GpD-(Enterococcus), Strep. GpC and GpG, Staph. aureus, Staph. epidermidis, Bacillus subtilis, Bacillus anthraxis, Listeria monocytogenes, Anaerobic cocci, Clostridium spp., and Actinomyces spp.

Antigens derived from pathogenic microorganisms typically activate the cell-mediated immune response when administered to a subject. Enhancing the immune response to such antigens, according to this embodiment of the present invention, therefore typically involves enhancing the cell-mediated immune (CMI) response to the pathogen. Thus, for example, when the immunoeffector is an antigen derived from a pathogenic microorganism, as described hereinabove, the method according to this aspect of the present invention can be beneficially used for providing the subject with a protective cell-mediated immune response to the pathogen and thus to any infectious or viral disease caused by the pathogen.

According to an embodiment of this aspect of the present invention, the immunoeffector is a T-cell independent antigen and the method according to this aspect of the present invention can be beneficially used for enhancing the T-cell independent immune response to the T-cell independent antigen.

Representative examples of T-cell independent antigens that can be used as immunoeffectors according to this embodiment of the present invention include, without limitation, carbohydrates (e.g. polysaccharides, lipopolysaccharides), lipids (e.g. liposomes), glycolipids, phosopholipids (e.g. phosphorylcholine), carrier conjugates (e.g. H. influenza conjugate vaccine, polysaccharide conjugate, lipid conjugate, phage conjugate), viruses (e.g., phages, such as T4 phage), parasites, fingi and yeast, and TI antigens recognized by immature T cells (e.g., CD1 molecules), such as lipoarabinomannan, which when administered to a subject activate the immune response without interacting with the T-lymphocytes.

Polysaccharides which may be used as immunoeffectors according to this embodiment of the present invention include, without limitation, bacterial polysaccharides, such as bacterial capsular polysaccharides (e.g., Streptococcus pneumoniae capsular polysaccharide, such as the PNU-Immune 23 vaccine, Neisseria meningiditis A, C, Y and W-135 serogroups, Haemophilus influenzae, Brucella abortis), and bacterial gram-negative cell wall polysaccharides.

Further examples may be found in, for example, Bondada and M. Grag, “Thymus-Independent Antigens” in The Handbook of B and T Lymphocytes, E. Charles Snow, Academic Press, Inc., San Diego, (1994) pages 343-370), which is incorporated by reference as if fully set forth herein

Enhancing the immune response to such antigens, according to this embodiment of the present invention, typically do not involve enhancement of CMI response but rather involves enhancement of the humoral immune response of the subject, resulting in an enhanced IgG2a antibody response.

Hence, when the immunoeffector is a T-cell independent antigen, as described hereinabove, the method according to this aspect of the present invention can be beneficially used for providing the subject with an enhanced humoral immune response to a T-cell independent antigen, resulting in an enhanced IgG2a antibody response, mediated by IL-12, and thus with enhanced protection against T-cell independent antigens, as discussed above.

When the antigen is a T-cell dependent antigen, enhancement of the immune response may comprise enhancing a type 1/Th1 T cell immune response. Non-limiting examples of T-cell dependent antigens that stimulate such an immune response include lipopeptides, such as, for example, a lipoprotein of Mycobacterium tuberculosis, the lipopeptide antigen that is central to an effective cell-mediated immune response to intracellular pathogens or interferon-γ-sensitive tumors.

Enhancing a type 1/Th1 T cell immune response is useful, by way of example, where peripheral blood mononuclear cells are in need of inducing to produce IL-12; in vivo where a subject is in need of enhancement of the type 1/Th1 cell response for enhanced cell-mediated immunity; and ex vivo where body fluids, such as blood or bone marrow, may be removed from a body and treated with the appropriate immunoeffector along with a tellurium-containing compound with resultant enhanced cell-mediated immunity.

In another embodiment of this aspect of the present invention, the immunoeffector is an antigen derived from a cancer cell or a cancer cell transfected with a selected antigen, and the method according to this embodiment of the present invention comprises eliciting the host's cell mediated immune response against the cancer cell or the cancer cell transfected with the selected antigen. For example, the method according to this embodiment of the present invention may be effected by co-administering any purified tumor antigen with the tellurium-containing compound. Alternatively, the method may involve use of an antigen that normally is not expressed on a cancer cell. The selected antigen is transferred into the cancer cell and the transfected cell itself, expressing the antigen, is used as an immunoeffector or as a therapeutic. Such a method results in an enhanced proliferative effect on T cells and increased production of cytokines, such as interleukin-12 and interferon-γ, thereby increasing the immune response to the cancer cell.

According to another embodiment of the present invention, the immunoeffector is an antigen-releasing agent.

The phrase “antigen-releasing agent” describes a biomolecule which releases antigen from the cell membrane of an antigen-presenting cell.

An example of an antigen-releasing agent is a chemotherapeutic agent, which when administered to a subject kills cancer cells, which, as a result, release antigens to the cancer.

Hence, according to a preferred embodiment of the present invention, the immunoeffector is a chemotherapeutic agent, and the method, according to this aspect of the present invention comprises enhancing the immune response to the chemotherapeutic agent.

A non-limiting example of a chemotherapeutic agent suitable for use in the method of this aspect of the invention is cyclophosphamide.

As is discussed hereinabove and is further demonstrated in the Examples section that follows, it was found that the direct effect of the tellurium-containing compounds described herein when contacted with various immune cells, is enhancement of the IL-12 production.

As is further discussed hereinabove, enhancement of IL-12 production is associated with enhancing the immune response to various antigens and other immunoeffectors.

Hence, according to a further aspect of the present invention there is provided a method for enhancing interleukin-12 production in a subject having a condition in which enhanced immune response induced by an immunoeffector is beneficial. The method, according to this aspect of the present invention, is effected by administering to the subject an effective adjuvanting amount of at least one tellurium-containing compound as described herein.

The method according to this aspect of the present invention can be effectively used for efficiently providing a subject with an adjuvant for use in the treatment of various medical conditions, including, without limitation, cancer (including leukemia and solid tumors, such as adrenal tumors, bone tumors, gastrointestinal tumors, brain tumors, breast tumors, skin tumors, lung tumors, ovarian tumors, and genitourinary tumors), immune deficiencies (such as HIV positive or Acquired Immunodeficiency Syndrome (AIDS)), autoimmune diseases and infectious diseases, using amounts of the tellurium-containing compounds that are effective in each condition.

Since interleukin 12 (IL-12) is an important regulatory cytokine that has a function central to the initiation and regulation of immune responses, the immunoenhancing effect of the tellurium-containing compound via IL-12 may be based on the activation of both innate and adaptive immune systems. For example, in cancer patients, IL-12 has an anti-tumor effect based on the activation of both innate and antigen-specific adaptive immunity against tumor cells and the ability to inhibit tumor angiogenesis through INF-γ. In immune deficiencies, the tellurium-containing compound enhances deficient cell-mediated immunity, possibly by restoration of impaired IL-12 production.

As used herein, the term “cancer” describes a group of diseases characterized by uncontrolled cell division leading to growth of abnormal tissue. These include, for example, leukemia and solid tumors that arise spontaneously, by contact with a carcinogenic agent, by irradiation or by oncoviruses. These conditions are well known to those who are skilled in the art and include, without limitation, adrenal tumors, bone tumors, gastrointestinal tumors, brain tumors, breast tumors, skin tumors, lung tumors, ovarian tumors, genitourinary tumors and the like. The Merck Manual 13th Edition, Merck & Co. (1977) describes many of these conditions (see, for example, pages 647-650; 828-831; 917-920; 966; 970-974; 1273, 1277, 1371-1376; 1436-1441; 1563; 1612-1615 of the publication, which are incorporated by reference as if fully set forth herein).

As used herein, the phrase “immunodeficiency diseases” describes a diverse group of conditions characterized chiefly by an increased susceptibility to various infections with consequent severe acute, recurrent and chronic disease which result from one or more defects in the specific or nonspecific immune systems (for an exemplary list of such conditions, see pages 205-220 of the Merck Manual 13th Edition describe, which are incorporated by reference as if fully set forth herein). The most representative example of an immunodeficiency disease is Acquired Immunodeficiency Syndrome (AIDS).

As used herein, the phrase “autoimmune diseases” includes disorders in which the immune system produces autoantibodies to an endogenous antigen, with consequent injury to tissues. Examples of such conditions can be found in pages 241-243 of the Merck Manual 13th Edition, which are incorporated by reference as if fully set forth herein.

The phrase “infectious diseases” includes those pathologic conditions that arise from a pathogenic organism (e.g., bacterial, viral or fungus organisms) that invades and disrupts the normal function of the mammalian body. Pages 3-149 of the Merck Manual 13th Edition describe these conditions and they are incorporated herein by reference.

According to a further aspect of the present invention there is provided a tellurium-containing compound for use as an adjuvant in immunization, for enhancing the immune response of a subject to an immunoeffector.

According to still a further aspect of the present invention, there are provided novel vaccine compositions and methods of adjuvantation of vaccines intended to provide a protective cell-mediated or humoral immune response to an immunoeffector, using as an adjuvant, a tellurium-containing compound.

When used as an adjuvant for a selected vaccine composition containing an antigen of a pathogenic microorganism, the tellurium-containing compound is preferably admixed as part of the vaccine composition itself.

Hence, according to yet a further aspect of the present invention, there is provided a pharmaceutical composition which comprises an immunoeffector, as is detailed hereinabove, one or more of the tellurium-containing compounds described herein and a pharmaceutically acceptable carrier.

Such a pharmaceutical composition can be used as a highly effective vaccine composition against various medical conditions, as is detailed hereinabove.

As used herein a “pharmaceutical composition” refers to a preparation of one or more of the active ingredients (herein, an immunoeffector and a tellurium-containing compound) with other chemical components such as physiologically suitable carriers and excipients. The purpose of a pharmaceutical composition is to facilitate administration of a compound or a mixture of compounds to the subject treated.

Hereinafter, the phrases “physiologically acceptable carrier” and “pharmaceutically acceptable carrier” which may be interchangeably used refer to a carrier or a diluent that does not cause significant irritation to the subject and does not abrogate the biological activity and properties of the administered compound.

Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Suitable pharmaceutical excipients include without limitation, calcium carbonate, calcium phosphate, various sugars and types of starch, cellulose derivatives, gelatin, vegetable oils, polyethylene glycols, sodium stearate, glycerol monostearate, talc, sodium chloride, glycerol, propyleneglycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The pharmaceutical compositions herein described may also comprise suitable solid or gel phase carriers or excipients. Examples of such carriers or excipients include, but are not limited to, calcium carbonate, calcium phosphate, various sugars, starches, cellulose derivatives, gelatin and polymers such as polyethylene Pharmaceutical compositions of the present invention may be manufactured by processes well known in the art, e.g., by means of conventional mixing, dissolving, granulating, dragee-making, levigating, emulsifying, encapsulating, entrapping or lyophilizing processes, utilizing an immunoeffector and a tellurium-containing compound as described herein.

Further techniques for formulation and administration of active ingredients may be found in “Remington's Pharmaceutical Sciences,” Mack Publishing Co., Easton, Pa., latest edition, which is incorporated herein by reference as if fully set forth herein.

Pharmaceutical compositions for use in accordance with the present invention thus may be formulated in conventional manner using one or more pharmaceutically acceptable carriers comprising excipients and auxiliaries, which facilitate processing of the active ingredients into preparations which, can be used pharmaceutically. Proper formulation is dependent upon the route of administration chosen.

Formulations for oral delivery can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should be suitable for the mode of administration.

For oral administration, the active ingredients can be formulated readily by combining the active ingredients with pharmaceutically acceptable carriers well known in the art. Such carriers enable the active ingredients of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For administration by inhalation, the ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the active ingredient and a suitable powder base such as lactose or starch.

The active ingredients described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

The composition can be formulated as rectal compositions such as suppositories or retention enemas, using, e.g., conventional suppository bases such as cocoa butter or other glycerides.

The dosage may vary depending upon the dosage form employed and the route of administration utilized. The exact formulation, route of administration and dosage can be chosen individually.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions according to the present invention formulated in a compatible pharmaceutical carrier may also be prepared, placed in an appropriate container, and labeled for use in immunization.

In any of the aspects described herein, the phrase “tellurium-containing compound” encompasses any compound that includes one or more tellurium atoms and exhibits immunomodulating properties.

The phrase “immunomodulating properties” includes any effect of the compound on the immune response of a subject. Exemplary immunomodulating properties can be manifested, for example, by an effect on cytokines secretion, interleukins production, lymphocytes function, and the like. Preferably, the immunomodulating property is stimulation of IL-12 production.

The compounds described herein preferably comprise at least one tellurium dioxide moiety.

Thus, the compound can be, for example, an inorganic tellurium-containing compound such as, for example, tellurium dioxide (TeO₂) per se.

The compound can alternatively be an organic tellurium-containing compound which includes one or more tellurium atoms and one or more organic moieties that are attached thereto.

Representative examples of inorganic tellurium-containing compounds that were shown to exert immunomodulating properties and hence are particularly useful in the context of the present invention include, for example, TeO₂.

Also included are compounds that form TeO₂ in aqueous solutions, preferably in the form of an organic complex such as, for example, a TeO₂ complex with citric acid or ethylene glycol. A representative example of the latter is the complex TeO₂.HOCH₂CH₂OH.NH₄Cl.

Organic tellurium-containing compounds that were shown to exert immunomodulating properties and hence are particularly useful in the context of the present invention include, for example, ammonium salts, or any other salts, of halogenated tellurium-containing compounds having a bidentate cyclic moiety attached to the tellurium atom. The bidentate cyclic moiety is preferably a di-oxo moiety having two oxygen atoms attached to the tellurium atom. Alternatively, the bidentate cyclic moiety can be a di-thio moiety, in which two sulfur atoms are attached to the tellurium atom.

Preferred compounds in this category are collectively represented by the general Formula I:

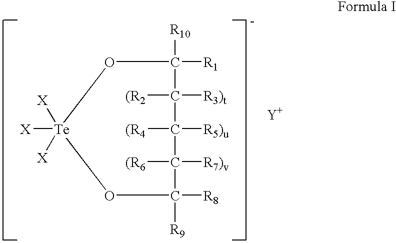

In the general Formula I above, each of t, u and v is independently 0 or 1, such that the compound may include a five-membered ring, a six-membered ring, or a seven-membered ring. Preferably, each of t, u and v is 0, such that the compound includes a five-membered ring.

X is a halogen atom, as described hereinabove, and is preferably chloro.

Y is selected from the group consisting of ammonium, phosphonium, potassium, sodium and lithium, and is preferably ammonium.

Each of R₁-R₁₀ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 5 carbon atoms. The alkyl group may be substituted or unsubstituted. When substituted, the substituent group can be, for example, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfate, cyano, nitro, sulfonamide, phosphonyl, phosphinyl, carbonyl, thiocarbonyl, carboxy, thiocarboxy, carbamate, thiocarbamate, amido, sulfonamido, and amino, as these terms are defined herein.

As used herein, the term “hydroxyalkyl” refers to an alkyl, as this term is defined herein, substituted by a hydroxy group, as defined herein, and includes, for example, hydroxymethyl, hydroxyethyl, hydroxypropyl and hydroxy-n-butyl.

The term “haloalkyl” refers to an alkyl, as this term is defined herein, substituted by a halogen, as defined herein, and includes, for example, chloromethyl, 2-iodoethyl, 4-bromo-n-butyl, iodoethyl, 4-bromo-n-pentyl and the like.

The term “alkanoyloxy” refers to a carbonyl group, as define herein and includes, for example, acetyl, propionyl, butanoyl and the like.

The term “carboxyalkyl” refers to an alkyl, as this term is defined herein, substituted by a carboxy group, as defined herein, and includes, for example, carboxymethyl, carboxyethyl, ethylenecarboxy and the like.

The term “alkylcarbonylalkyl” refers to an alkyl, as this term is defined herein, substituted by a carbonyl group, as defined herein, and includes, for example, methanoylmethyl, ethanoylethyl and the like.

The term “amidoalkyl” refers to an alkyl, as this term is defined herein, substituted by an amide group, as defined herein, and includes, for example, —CH₂CONH₂; —CH₂CH₂CONH₂; —CH₂CH₂CH₂CONH₂ and the like.

The term “cyanoalkyl” refers to an alkyl, as this term is defined herein, substituted by an cyano group, as defined herein, and includes, for example, —CH₂CN; —CH₂CH₂CN; —CH₂CH₂CH₂CN and the like.

The term “N-monoalkylamidoalkyl” refers to an alkyl, as this term is defined herein, substituted by an amide group, as defined herein, in which one of R′ and R″ is an alkyl, and includes, for example, —CH₂CH₂CONHCH₃, and —CH₂CONHCH₂CH₃.

The term N,N-dialkylamidoalkyl refers to an alkyl, as this term is defined herein, substituted by an amide group, as defined herein in which both R′ and R″ are alkyl, and includes, for example, —CH₂CON(CH₃)₂; CH₂CH₂CON(CH₂—CH₃)₂ and the like.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, phosphonyl, phosphinyl, carbonyl, thiocarbonyl, carboxy, thiocarboxy, carbamate, thiocarbamate, amido, sulfonamido, and amino, as these terms are defined herein.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfate, cyano, nitro, phosphonyl, phosphinyl, phosphonium, carbonyl, thiocarbonyl, carboxy, thiocarboxy, carbamate, thiocarbamate, amido, sulfonamido, and amino, as these terms are defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfate, cyano, nitro, phosphonyl, phosphinyl, phosphonium, carbonyl, thiocarbonyl, carboxy, thiocarboxy, carbamate, thiocarbamate, amido, sulfonamido, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfate, cyano, nitro, phosphonyl, phosphinyl, phosphonium, carbonyl, thiocarbonyl, carboxy, thiocarboxy, carbamate, thiocarbamate, amido, sulfonamido, and amino, as these terms are defined herein. Representative examples are piperidine, piperazine, tetrahydro furane, tetrahydropyrane, morpholino and the like.

A “hydroxy” group refers to an —OH group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein for R′.

A “carboxy” group refers to a —C(═O)—O—R′ or a —O—C(═O)—R′ group, where R′ is as defined herein.

A “sulfinyl” group refers to an —S(═O)—R′ group, where R′ is as defined herein.

A “sulfonyl” group refers to an —S(═O)₂—R′ group, where R′ is as defined herein.

A “sulfate” group refers to a —O—S(═O)₂—OR′ group, where R′ is as defined herein.

A “sulfonamido” group refers to a —S(═O)₂—NR′R″ group or a R′S(═O)₂—NR″, with R′ is as defined herein and R″ is as defined for R′.

A “carbamyl” or “carbamate” group refers to an —OC(═O)—NR′R″ group or a R″OC(—O)—NR′— group, where R′ and R″ are as defined herein.

A “thiocarbamyl” or “thiocarbamate” group refers to an —OC(═S)—NR′R″ group or an R″OC(═S)NR—′ group, where R′ and W″ are as defined herein.

An “amino” group refers to an —NR′R″ group where R′ and R″ are as defined herein.

An “amido” group refers to a —C(—O)—NR′R″ group or a R′C(═O)—NR″ group, where R′ and R″ are as defined herein.

A “nitro” group refers to an —NO₂ group.

A “cyano” group refers to a —C≡N group.

The term “phosphonyl” describes a —O—P(═O)(OR′)(OR″) group, with R′ and R″ as defined hereinabove.

The term “phosphinyl” describes a —PR′R″ group, with R′ and R″ as defined hereinabove.

As cited hereinabove, the compounds in this category are salts of organic tellurium-containing compounds. The salts can be, for example, ammonium salts, phosphonium salts and alkaline salts such as potassium salts, sodium salts, lithium salts and the like.

Hence, Y in Formula I above can be a phosphonium group, as defined herein, an ammonium group, as defined herein, potassium (K⁺), sodium (Na⁺) or lithium (Li⁺).

As used herein, the term “phosphonium” describes a —P⁺R′R″R′″group, with R′ and R″ as defined herein and R′″ is as defined for R′. The term “phosphonium”, as used herein, further refers to a —P⁺R₆ group, wherein each of the six R substituents is independently as defined herein for R, R″ and R′″.

The term “ammonium” describes a —N⁺R′R″R′″ group, with R′, R″ and R′″ as defined herein.

More preferred compounds in this category include compounds having the general Formula I described above, in which Y is ammonium or phosphonium, t, u and v are each 0, and each of R₁, R₈, R₉ and R₁₀ is independently hydrogen or alkyl. These compounds can be represented by the following structure:

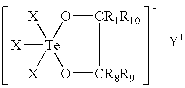

wherein each of R₁, R₈, R₉ and R₁₀ is independently hydrogen or alkyl, preferably methyl, and X is halogen, preferably chloro.

The presently most preferred compound of formula I for use in the context of the present invention has the following structure:

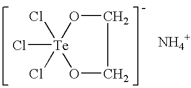

This compound is ammonium trichloro(dioxyethylene-O,O′)tellurate, which is also referred to herein and in the art as AS101.

Additional representative examples of organic tellurium-containing compound that are suitable for use in the context of the present invention include halogenated tellurium having a bidentate cyclic moiety attached to the tellurium atom. The bidentate cyclic moiety is preferably a di-oxo ligand having two oxygen atoms attached to the tellurium atom. Alternatively, the bidentate cyclic moiety can be a di-thio ligand, in which two sulfur atoms are attached to the tellurium atom.

Preferred compounds in this category can be represented by the general Formula II:

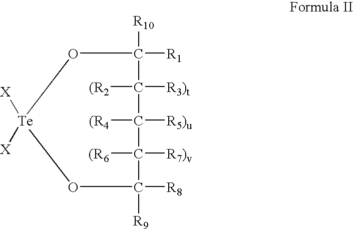

wherein t, u, v, X and R₁-R₁₀ are as defined hereinabove.

More preferred compounds are those in which t, u, and v are each 0, and X is chloro, such as, but not limited to, the compound having the following structure:

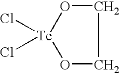

The above compound is also known and referred to herein as AS103.

The organic tellurium-containing compounds having Formulae I and II can be readily prepared by reacting tetrahalotelluride such as TeCl₄ with a dihydroxy compound, as is described in detail in U.S. Pat. Nos. 4,752,614, 4,761,490, 4,764,461 and 4,929,739.

Additional representative examples of organic tellurium-containing compound that are suitable for use in the context of the present invention include compounds in which two bidentate cyclic moieties are attached to the tellurium atom. Preferably, each of the cyclic moieties is a di-oxo moiety. Alternatively, one or more of the cyclic moieties is a di-thio moiety.

Preferred compounds in this category are collectively represented by the general Formula III:

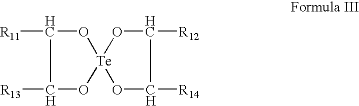

wherein each of R₁₁-R₁₄ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido, as these terms are defined herein.

More preferred compounds in this category are those in which each of R₁₁-R₁₄ is hydrogen.

Additional representative examples of organic tellurium-containing compounds that are suitable for use in the context of the present invention include the recently disclosed bis-tellurium compounds having general Formula IV:

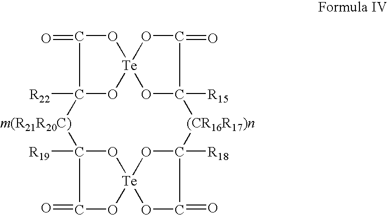

wherein each of R₁₅-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido, as these terms are defined herein; and

m and n are each an integer from 0 to 3.

Preferred compounds in this category are those in which m and n are each 0.

The presently most preferred compound in this family is a compound referred to herein as SAS, in which R₁₅, R₁₈, R₁₉ and R₂₂ are all hydrogen, and which has the following structure:

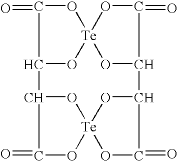

Compounds having the general Formula IV can be readily prepared by reacting substantially equimolar amounts of a tellurium tetralkoxide and a polycarboxylic acid. These materials are combined in the presence of a water free organic solvent such as dried ethanol, dimethyl sulfoxide, i-propanol and the like. Generally the reaction may take place at ambient conditions but if desired higher or lower temperatures and higher or lower pressures may be utilized.

Exemplary tellurium tetraalkoxide compounds that are usable in the preparation of the compounds having general Formula IV above include, without limitation, tetramethoxide, tetraethoxide, tetrapropoxide, tetraisopropoxide, tetrabutoxide, and tetrapentoxide tellerium compounds.

Useful polycarboxylic acids include also polyhydroxy polycarboxylic and hydroxy polycarboxylic acids. Exemplary polycarboxylic acids that are usable in the preparation of the compounds having general Formula IV above include, without limitation, tartaric acid, glutaric acid, succinic acid, malonic acid, gluconic acid and the like.

Additional organic tellurium-containing compounds that are suitable for use in the context of the present invention include those having the general Formula V:

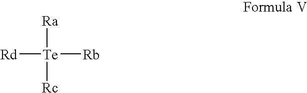

wherein each of Ra, Rb, Rc and Rd is independently selected from the group consisting of halogen alkyl, aryl, cycloalkyl, alkoxy, aryloxy, thioalkoxy, thioaryloxy, carboxy, carbonyl, thiocarboxy, thiocarbonyl, carbamyl, and thiocarbamyl, as these terms are defined hereinabove, whereby at least one of Ra-Rd is not halogen, namely, is selected from the group consisting of alkyl, aryl, cycloalkyl, alkoxy, aryloxy, thioalkoxy, thioaryloxy, carboxy, carbonyl, thiocarboxy, thiocarbonyl, carbamyl, and thiocarbamyl.

Compounds in this category include those in which one of Ra, Rb, Rc and Rd is halogen alkyl, aryl, cycloalkyl, alkoxy, aryloxy, thioalkoxy, thioaryloxy, carboxy, carbonyl, thiocarboxy, thiocarbonyl, carbamyl, or thiocarbamyl, whereby the others halogen atoms, e.g., chloro.

Other compounds in this category include those in which two or three of Ra, Rb, Rc and Rd are as described above and the others are halogens e.g., chloro.

Other compounds in this category include those in which each of Ra, Rb, Rc and Rd is as described hereinabove.

According to a further aspect of the present invention there is provided a pharmaceutical composition, which comprises the tellurium-containing compound, the immunoeffector and a pharmaceutically acceptable carrier.

The compounds described above can be administered or otherwise utilized in this and other aspects of the present invention, either as is or as a pharmaceutically acceptable salt thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

Effect of AS101 on IL-12 Production by Human Monocytes

Adherent Peripheral Blood Mononuclear Cells (PBMCs) from a tuberculin-negative healthy donor were incubated with AS101 or AS103 (0.5-2 μg/ml PBS) or E. coli lipopolysaccharide (LPS) (1 ng/ml PBS; Sigma) for 24 hours. Supernatants were collected after 28 hours for analysis of IL-12 production. Cell supernatants were determined using commercially available Enzyme-Linked Immunosorbent Assay (ELISA) kits (R&D Systems). Supernatants were tested for IL-12p40 by ELISA kit (Endogene).

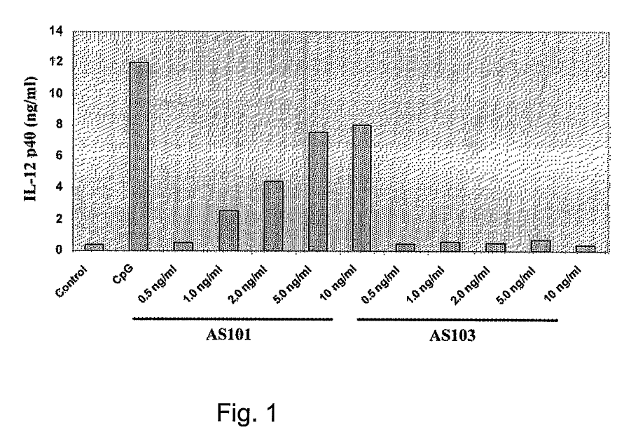

The data obtained, presented in FIG. 1, clearly indicates that AS101 is a potent inducer of IL-12 p40 production in freshly isolated peripheral blood human monocytes, in contrast to AS 103 which elicited a very low response.

### Example 2

### Effects of AS101 on IL-12 p40 Production by Murine Bone Marrow-Derived Dendritic Cells

Murine bone marrow-derived dendritic cells (DC) were prepared by culturing bone marrow cells from the femur and tibia of mice in RPMI medium supplemented with 10% supernatant from a granulocyte-monocyte colony-stimulating factor-secreting cell line.

On day 7 of culture, cells were collected, washed, and resuspended in RPMI medium. DC (10⁶ cells/ml) were cultured with AS101 or AS103 (0.5-10 μg/ml PBS), or with CpG. Supernatants were collected after 24 hours for analysis of IL-12 p40 production. Cell supernatants were determined using commercially available ELISA kits (R&D Systems).

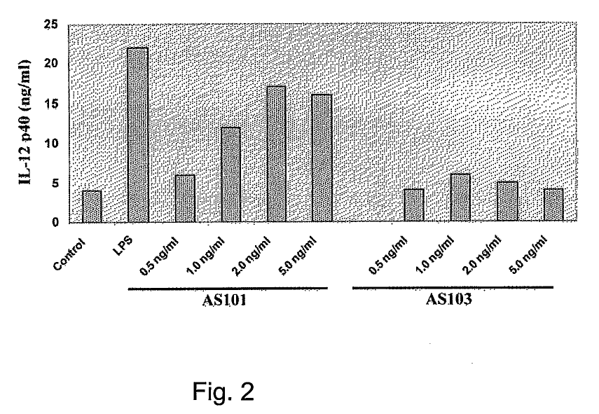

The data obtained is presented in FIG. 2 and clearly indicates that AS101 is a potent inducer of IL-12 p40 production in bone marrow-derived dendritic cells.

### Example 3

### Effects of AS101 on Serum Antibody Responses to KLH

Serum was obtained from mice immunized with depyrogenated keyhole limpet hemocyanin (KLH) (5 μg; Calbiochem, La Jolla, Calif.); with KLH plusphosphorothioate-stabilized oligodeoxynucleotide-containing CpG motifs (CpG-ODN) (5′-GCTAGACGTTAGCGT-3′), synthesized by Sigma-Genosys Ltd., Cambridge, United Kingdom; with KLH, plus CpG, plus AS101 (10 μg/ml PBS); or with Dulbecco's PBS alone (Sigma, Poole, United Kingdom), each in a final volume of 50 μl PBS.

On day 7 after immunization, mice were sacrificed by cervical dislocation, and serum and popliteal lymph nodes were collected. Titers of KLH-specific IgG1 and IgG2a in the serum of the immunized mice were determined by ELISA, and analysed for the presence of antibody subclasses IgG1 and IgG2a.

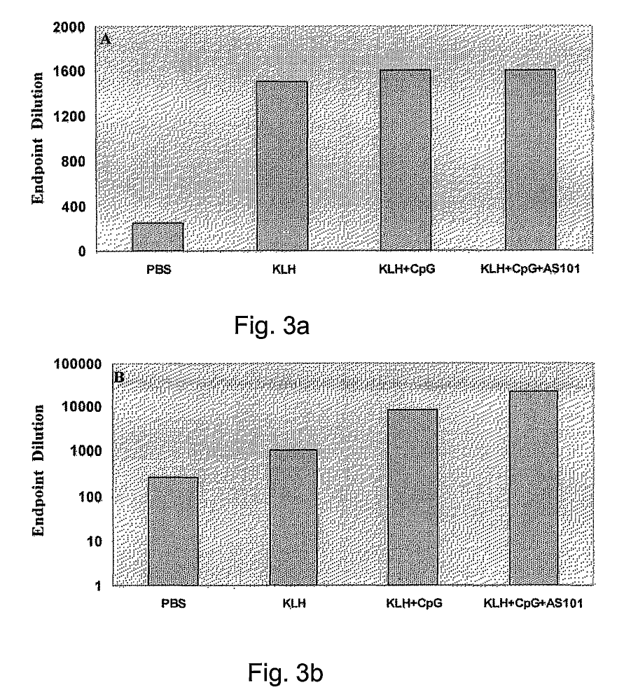

As seen in FIGS. 3a and 3b, production of both IgG1 and IG2a antibodies was elicited by immunization with KLH. The antibody titer was increased by the use of CpG together with the KLH. However, FIG. 3a shows that no further increase in the IgG1 response to KLH plus CpG was elicited by the addition of AS101. In contrast, as seen in FIG. 3b, production of IgG2a in response to KLH plus CpG was significantly increased by the addition of AS101.

The functional properties of Ig are closely related to their isotype. For instance, IgG2a antibodies activate the complement system more readily than do IgG1 antibodies (Klaus et al., Immunology 38:687, 1979); they bind to specific Fc receptors that are expressed on murine macrophages and are involved in phagocytosis (Heusser et al., J. Exp. Med. 145:1316, 1977); and they are quite efficient mediators of antibody-dependent cell-mediated cytotoxicity (Kipps et al., J. Exp. Med. 161:1, 1985).

Since IL-12 is known to be a potent stimulator of IFN-γ production which, among other activities, is a potent inducer of in vitro IgG2a secretion by activated B lymphocytes (Snapper et al., Science 236:944, 1987), including B cells stimulated after viral infection (Coutelier et al., J. Virol. 64:5383, 1990), these results strongly suggest a pathway involving induction of IL-12, resulting in IFN-γ production.

### Example 4

### Effect of SAS on IL-12 Production by Human Monocytes

Adherent Peripheral Blood Mononuclear Cells (PBMCs) from a tuberculin-negative healthy donor are incubated with SAS, in amounts equimolar to those described above for AS101 (0.87-3.5 μg/ml PBS) or E. coli lipopolysaccharide (LPS) (1 ng/ml PBS; Sigma) for 24 hours. Supernatants are collected after 28 hours for analysis of IL-12 production. Cell supernatants are determined using commercially available Enzyme-Linked Immunosorbent Assay (ELISA) kits (R&D Systems). Supernatants are tested for IL-12p40 by ELISA kit (Endogene).

### Example 5

Effects of SAS on IL-12 p40 Production by Murine Bone Marrow-Derived Dendritic Cells

Murine bone marrow-derived dendritic cells (DC) are prepared by culturing bone marrow cells from the femur and tibia of mice in RPMI medium supplemented with 10% supernatant from a granulocyte-monocyte colony-stimulating factor-secreting cell line.

On day 7 of culture, cells are collected, washed, and resuspended in RPMI medium. DC (10⁶ cells/ml) are cultured with SAS (0.87-17.5 μg/ml) or with CpG. Supernatants are collected after 24 hours for analysis of IL-12 p40 production. Cell supernatants are determined using commercially available ELISA kits (R&D Systems).

### Example 6

### Effects of SAS on Serum Antibody Responses to KLH

Serum was obtained from mice immunized with depyrogenated keyhole limpet hemocyanin (KLH) (5 μg; Calbiochem, La Jolla, Calif.); or with KLH plus phosphorothioate-stabilized oligodeoxynucleotide-containing CpG motifs (CpG-ODN) (5′-GCTAGACGTTAGCGT-3′), synthesized by Sigma-Genosys Ltd., Cambridge, United Kingdom; or with KLH, plus CpG, plus SAS (17.5 μg/ml PBS); or with Dulbecco's PBS (Sigma, Poole, United Kingdom) in a final volume of 50 μl.

On day 7 after the first or second immunization, mice are sacrificed by cervical dislocation, and serum and popliteal lymph nodes collected in the presence or absence of SAS (17.5 μg/ml). Titers of KLH-specific IgG1 and IgG2a in the serum of immunized mice are determined by ELISA, and analysed for the presence of antibody subclasses IgG1 and IgG2a.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-91. (canceled)

92. A method for enhancing the immune response of a subject to an immunoeffector, the method comprising administering to the subject an amount of the immunoeffector and an adjuvanting effective amount of at least one tellurium-containing compound selected from the group consisting of tellurium dioxide (TeO₂), a complex of TeO₂, a compound having general Formula I: 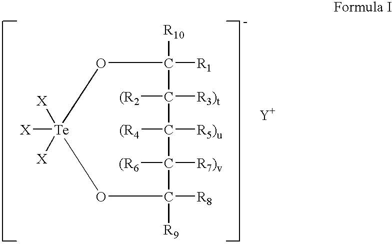
a compound having general Formula II: 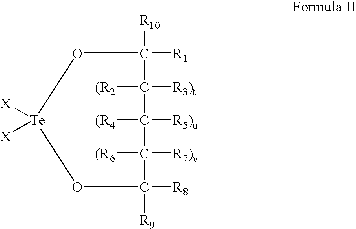
a compound having general Formula III: 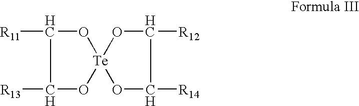
and
a compound having general Formula IV: 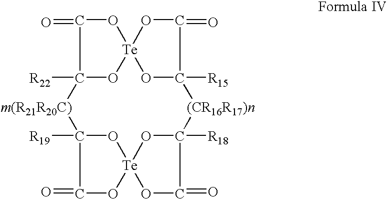
wherein:
each of t, u and v is independently 0 or 1;
each of m and n is independently an integer from 0 to 3;
Y is selected from the group consisting of ammonium, phosphonium, potassium, sodium and lithium;
X is a halogen atom; and
each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido.

93. A method of enhancing interleukin-12 production in a subject having a condition in which enhanced immune response induced by an immunoeffector is beneficial, the method comprising administering to the subject an effective adjuvanting amount of at least one tellurium-containing compound selected from the group consisting of tellurium dioxide (TeO₂), a complex of TeO₂, a compound having general Formula I: 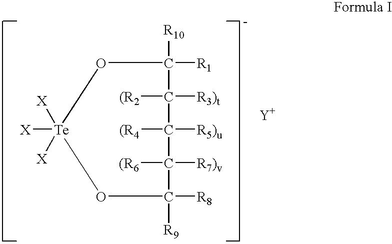
a compound having general Formula II: 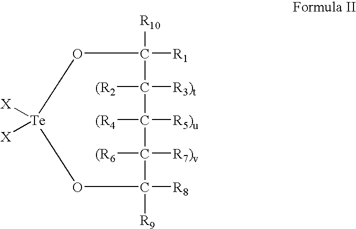
a compound having general Formula III: 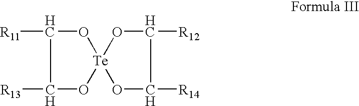
and
a compound having general Formula IV: 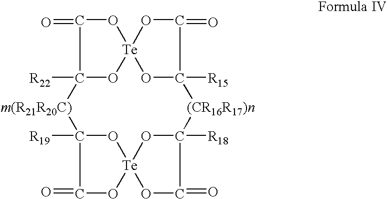
wherein:
each of t, u and v is independently 0 or 1;
each of m and n is independently an integer from 0 to 3;
Y is selected from the group consisting of ammonium, phosphonium, potassium, sodium and lithium;
X is a halogen atom; and
each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido.

94. The method of claim 93, wherein said condition is selected from the group consisting of cancer, an immune deficiency, an autoimmune disease, a viral disease and an infectious disease.

95. The method of claim 92, wherein said immunoeffector is an antigen.

96. The method of claim 93, wherein said immunoeffector is an antigen.

97. The method of claim 92, wherein said immunoeffector is a chemotherapeutic agent.

98. The method of claim 93, wherein said immunoeffector is a chemotherapeutic agent.

99. The method of claim 92, wherein said enhancing said immune response comprises stimulating interleukin-12 production in a host in response to said immunoeffector.

100. The method of claim 92, wherein said immunoeffector is an antigen derived from a cancer cell.

101. The method of claim 93, wherein said immunoeffector is a chemotherapeutic agent.

102. The method of claim 92, wherein said immunoeffector is a T-cell independent antigen and said enhancing said immune response comprises enhancing a T-cell independent immune response to said T-cell independent antigen.

103. The method of claim 93, wherein said immunoeffector is a T-cell independent antigen and said enhancing said immune response comprises enhancing a T-cell independent immune response to said T-cell independent antigen.

104. The method of claim 102, wherein said immune response is a humoral immune response.

105. The method of claim 92, wherein said immunoeffector is a lipopeptide.

106. The method of claim 93, wherein said immunoeffector is a lipopeptide.

107. The method of claim 92, wherein said tellurium-containing compound is selected from the group consisting of a compound having said general Formula I and a compound having said general Formula IV.

108. The method of claim 93, wherein said tellurium-containing compound is selected from the group consisting of a compound having said general Formula I and a compound having said general Formula IV.

109. The method of claim 92, wherein said at least one tellurium-containing compound forms a part of a pharmaceutical composition, said pharmaceutical composition further comprising said immunoeffector and a pharmaceutically acceptable carrier.

110. The method of claim 93, wherein said at least one tellurium-containing compound forms a part of a pharmaceutical composition, said pharmaceutical composition further comprising said immunoeffector and a pharmaceutically acceptable carrier.

111. The method of claim 109, wherein said tellurium-containing compound has said general Formula I, wherein t, u and v are each 0, each of R₁, R₈, R₉ and R₁₀ is hydrogen, X is chloro, Y is ammonium, and wherein a concentration of said at least one tellurium-containing compound ranges from about 0.5 μg to about 10 μg per 1 ml carrier.

112. The method of claim 110, wherein said tellurium-containing compound has said general Formula I, wherein t, u and v are each 0, each of R₁, R₈, R₉ and R₁₀ is hydrogen, X is chloro, Y is ammonium, and wherein a concentration of said at least one tellurium-containing compound ranges from about 0.5 μg to about 10 μg per 1 ml carrier.

113. The method of claim 109, wherein said tellurium-containing compound has said general Formula IV, wherein n and m are each 0, and R₁₅, R₁₈, R₁₉ and R₂₂ is hydrogen, and wherein a concentration of said at least one tellurium-containing compound ranges from about 0.0.2 μg to about 20 μg per 1 ml of said carrier.

114. The method of claim 110, wherein said tellurium-containing compound has said general Formula IV, wherein n and m are each 0, and R₁₅, R₁₈, R₁₉ and R₂₂ is hydrogen, and wherein a concentration of said at least one tellurium-containing compound ranges from about 0.0.2 μg to about 20 μg per 1 ml of said carrier.

115. A pharmaceutical composition comprising a pharmaceutically acceptable carrier, an immunoeffector and at least one tellurium-containing compound capable of enhancing a host's immune response to said immunoeffector, said at least one tellurium-containing compound being selected from the group consisting of tellurium dioxide (TeO₂), a complex of TeO₂, a compound having general Formula I: 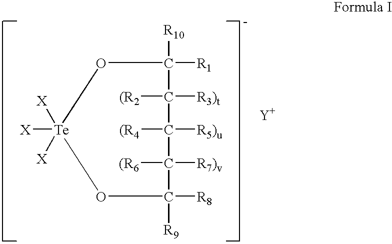
a compound having general Formula II: 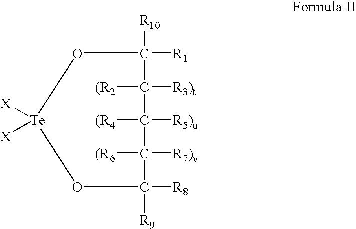
a compound having general Formula III: 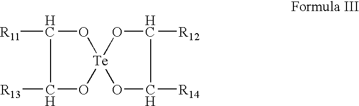
and
a compound having general Formula IV: 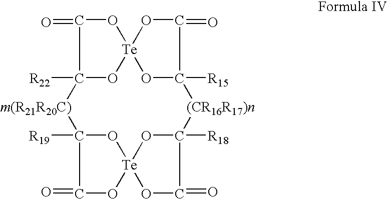
wherein:
each of t, u and v is independently 0 or 1;
each of m and n is independently an integer from 0 to 3;
Y is selected from the group consisting of ammonium, phosphonium, potassium, sodium and lithium;
X is a halogen atom; and
each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido.

116. The composition of claim 115, wherein said immunoeffector is an antigen.

117. The composition of claim 115, wherein said immunoeffector comprises a chemotherapeutic agent.

118. The composition of claim 115, wherein said immunoeffector is an antigen derived from a cancer cell.

119. The composition of claim 115, wherein said immunoeffector is a cancer cell transfected with a selected antigen.

120. The composition of claim 115, wherein said immunoeffector is a T-cell independent antigen.

121. The composition of claim 115, being identified for use as a vaccine composition.

122. The composition of claim 115, being identified for use in the treatment of a medical condition selected from the group consisting of a cancer, an immune deficiency, an autoimmune disease, a viral disease and an infectious disease.

123. The composition of claim 115, being identified for use in the treatment of a medical condition in which stimulation of interleukin-12 production is beneficial.

124. The composition of claim 123, wherein said medical condition is selected from the group consisting of cancer, an immune deficiency, an autoimmune disease, a viral disease and an infectious disease.

125. The composition of claim 115, wherein said tellurium-containing compound is selected from the group consisting of a compound having said general Formula I and a compound having said general Formula IV.
